(19) United States
(12) Patent Application Publication
(10) Pub. No.: US 2024/0358925 A1
(43) Pub. Date: Oct. 31, 2024

(54) DEVICES, SYSTEMS, AND METHODS FOR COMBINING AND/OR DELIVERING INJECTABLE MATERIALS

(71) Applicants: Boston Scientific Medical Device Limited, Galway (IE); Boston Scientific Scimed, Inc., Maple Grove, MN (US)

(72) Inventors: Nitesh Ghananil Baviskar, Kalyan West (IN); Benjamin Cleveland, Bellingham, MA (US); Sanjay Kumar Chandra, Pune (IN); Katie Knowles, Providence, RI (US); Junaid Mohammed Shaikh, Surat (IN); Marshall O'Hearn, Davenport, FL (US)

(73) Assignees: Boston Scientific Medical Device Limited, Galway (IE); Boston Scientific Scimed, Inc., Maple Grove, MN (US)

(21) Appl. No.: 18/646,175

(22) Filed: Apr. 25, 2024

Related U.S. Application Data
(60) Provisional application No. 63/462,146, filed on Apr. 26, 2023.

(51) Int. Cl. A61M 5/24; A61M 5/32

(52) U.S. Cl. CPC A61M 5/2448; A61M 5/2422; A61M 5/3293

(57) ABSTRACT

Mixing systems for producing a mixture to deliver to a treatment site. An illustrative system may comprise a needle hub, a multi-reservoir system, and a syringe. The multi-reservoir system may comprise a plunger assembly including a first tubular plunger and a second tubular plunger, a barrel portion including a housing defining a first reservoir in fluid communication with a first port and configured to contain a first constituent and a second annular reservoir configured to contain a second constituent and in fluid communication with a second port, and a third plunger. A third constituent may be injected from the syringe into the first reservoir to form a precursor. Actuating the plunger assembly relative to the barrel portion may cause the precursor and the second constituent to be delivered into the needle hub.

### CROSS-REFERENCE TO RELATED APPLICATIONS

This application claims the benefit of U.S. Provisional Patent Application Ser. No. 63/462,146 filed on Apr. 26, 2023, the disclosure of which is incorporated herein by reference.

### FIELD

The present disclosure relates generally to the field of devices for delivering injectable materials and/or compositions to a patient, and associated systems and methods. More particularly, the present disclosure relates to devices for combining constituents of injectable materials and/compositions, and associated systems and methods.

### BACKGROUND

Various forms of cancer and other medical conditions are treated by local application of radiation therapy. However, various risks may accompany radiation therapy. Since the conception of conformal radiotherapy, physicians have paid attention to the radiation dose delivered to the target and surrounding tissues. Investigators have been able to correlate side effects to the amount of tissue receiving a certain radiation dose. And yet, time, distance, and shielding affect the dose that is delivered. The less time an area is exposed to radiation, the less dose is delivered. The greater the distance from the radiation, the less dose is delivered. Filler materials may be injected into a treatment area to provide a shield to tissue surrounding the target of the radiation therapy. For instance, numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy. Various systems provide filler material to treatment sites to decrease the radiation dose to tissue surrounding radiation target sits (e.g., to shield the rectum during radiotherapy for prostate cancer). Such filler materials are often reactive, and therefore are generally combined/mixed immediately prior to or even during delivery to the patient.

Various systems are known for combining/mixing (e.g., in vitro) filler materials injected into radiation treatment areas. However, most such systems include numerous subcomponents, are complex to assemble, and are susceptible to filler mixing errors prior to delivery within a patient at a treatment site. Various challenges posed by such mixing systems may result in errors and mishaps which lead unnecessarily to increased procedure time and increased procedure costs. Solutions to these and other issues presented by combining and delivering injectable materials would be welcome in the art.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with certain embodiments of the present disclosure, devices, systems, and methods for combining and/or delivering injectable materials are disclosed.

In a first example, a system for producing a mixture to deliver to a treatment site may comprise a needle hub, a multi-reservoir system, and a syringe defining a lumen configured to contain a third constituent. The multi-reservoir system may comprise a plunger assembly including a first tubular plunger and a second tubular plunger, a barrel portion including a housing including an inner tubular wall defining a first reservoir in fluid communication with a first port and configured to contain a first constituent and an outer tubular wall together with the first wall defining a second annular reservoir in fluid communication with a second port and disposed about the first reservoir, the second annular reservoir configured to contain a second constituent, and a third plunger movably disposed relative to the plunger assembly and at least partially within the first reservoir. A distal end of the syringe may be configured to be releasably coupled to the first port such that actuation of the syringe causes the third constituent to be injected into the first reservoir to mix with the first constituent and form a precursor. Actuating the plunger assembly relative to the barrel portion may cause the precursor and the second constituent to be delivered into the needle hub.

In another example, a system for producing a mixture to deliver to a treatment site may comprise a needle hub, a multi-reservoir system, and a syringe defining a lumen configured to contain a third constituent. The multi-reservoir system may comprise a plunger assembly including a first tubular plunger and a second tubular plunger, a barrel portion including a housing including an inner tubular wall defining a first reservoir in fluid communication with a first port and configured to contain a first constituent and an outer tubular wall together with the first wall defining a second annular reservoir in fluid communication with a second port and disposed about the first reservoir, the second annular reservoir configured to contain a second constituent, and a third plunger movably disposed relative to the plunger assembly and at least partially within the first reservoir. A distal end of the syringe may be configured to be releasably coupled to the first port or the second port such that actuation of the syringe causes the third constituent to be injected into the first reservoir or the second reservoir to mix with the first or second constituent and form a precursor. Actuating the plunger assembly relative to the barrel portion causes the precursor containing the first and third constituents and the second constituent to be delivered into the needle hub, or causes the precursor containing the second and third constituents and the first constituent to be delivered into the needle hub.

Alternatively or additionally to any of the examples above, in another example, the system may further comprise a removable retainer positioned adjacent to a proximal end of the barrel portion, the removable retainer may prevent actuation of the plunger assembly relative to the barrel portion.

Alternatively or additionally to any of the examples above, in another example, the system may further comprise a removable retainer positioned between a proximal end of the barrel portion and a proximal end of the plunger assembly, the removable retainer may prevent actuation of the plunger assembly relative to the barrel portion.

Alternatively or additionally to any of the examples above, in another example, the system may further comprise a cap removably coupled with the first port of the barrel portion.

Alternatively or additionally to any of the examples above, in another example, the system may further comprise a plug removably coupled with the second port of the barrel portion.

Alternatively or additionally to any of the examples above, in another example, the first plunger and the second plunger may be concentrically arranged.

Alternatively or additionally to any of the examples above, in another example, the first reservoir and the second annular reservoir may be concentrically arranged.

Alternatively or additionally to any of the examples above, in another example, the plunger assembly may be configured to be actuated with the first port of the barrel portion free from the cap and the second port of the barrel portion free from the plug.

Alternatively or additionally to any of the examples above, in another example, the third plunger may be unsecured to both the plunger assembly and the barrel portion.

Alternatively or additionally to any of the examples above, in another example, the system may further comprise a needle that is configured to be coupled to the needle hub.

Alternatively or additionally to any of the examples above, in another example, the needle hub may comprise a first lumen in fluid communication with the first reservoir of the barrel portion via the first port, a second lumen in fluid communication with the second reservoir of the barrel portion via the second port, a central lumen configured to be in fluid communication with a needle, and a mixing region connecting the first and second lumens with the central lumen.

Alternatively or additionally to any of the examples above, in another example, the needle hub may be removably coupled to a distal end region of the barrel portion of the multi-reservoir system.

Alternatively or additionally to any of the examples above, in another example, the plunger assembly and the barrel portion may be assembled in a telescoping arrangement.

Alternatively or additionally to any of the examples above, in another example, the third plunger may be disposed at least partially within a lumen of the first tubular plunger.

In another example, a method for producing a mixture with a mixing system to deliver to a treatment site may comprise coupling a distal end of a syringe with a first port of the multi-reservoir system, injecting a first constituent into a first reservoir of the multi-reservoir system to mix the first constituent with a second constituent contained in the first reservoir of the multi-reservoir system to form a precursor, coupling a needle hub having a mixing region to a distal end of the multi-reservoir system, and actuating a plunger assembly to move the precursor and a third constituent disposed within a second reservoir of the multi-reservoir system into the mixing region of the needle hub to form an injectable mixture.

Alternatively or additionally to any of the examples above, in another example, the method may further comprise removing a retainer from the plunger assembly prior to actuating the plunger assembly.

Alternatively or additionally to any of the examples above, in another example, the method may further comprise actuating the plunger assembly prior to coupling the needle hub to the distal end of the multi-reservoir system to purge air from the multi-reservoir system.

Alternatively or additionally to any of the examples above, in another example, the method may further comprise removing a first cap or plug from a first port of the multi-reservoir system prior to coupling the distal end of the syringe with the first port of the multi-reservoir system.

Alternatively or additionally to any of the examples above, in another example, the method may further comprise removing a second cap or plug from a second port of the multi-reservoir system prior to coupling the needle hub to the distal end of the multi-reservoir system.

In another example, a kit for producing a mixture for delivery to a treatment site may comprise a multi-reservoir system, a syringe defining a lumen configured to contain a third constituent, and an injection system. The multi-reservoir system may comprise a plunger assembly including a first tubular plunger and a second tubular plunger, a barrel portion including a housing including an inner tubular wall defining a first reservoir in fluid communication with a first port and configured to contain a first constituent and an outer tubular wall together with the first wall defining a second annular reservoir in fluid communication with a second port and disposed about the first reservoir, the second annular reservoir configured to contain a second constituent, and a third plunger movably disposed relative to the plunger assembly and at least partially within the first reservoir. The injection system may comprise a needle hub and a needle coupled to the needle hub.

Alternatively or additionally to any of the examples above, in another example, the kit may further comprise a removable retainer positioned adjacent to a proximal end of the barrel portion, the removable retainer configured to prevent actuation of the plunger assembly relative to the barrel portion.

Alternatively or additionally to any of the examples above, in another example, the kit may further comprise a removable retainer positioned between a proximal end of the barrel portion and a proximal end of the plunger assembly, the removable retainer configured to prevent actuation of the plunger assembly relative to the barrel portion.

Alternatively or additionally to any of the examples above, in another example, the kit may further comprise a cap removably coupled with the first port of the barrel portion.

Alternatively or additionally to any of the examples above, in another example, the kit may further comprise a plug removably coupled with the second port of the barrel portion.

Alternatively or additionally to any of the examples above, in another example, the kit may further comprise a connector, the connector configured to couple the needle hub to a saline syringe.

Alternatively or additionally to any of the examples above, in another example, the third plunger may be disposed at least partially within a lumen of the first tubular plunger.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to facilitate injection into a patient. For purposes of clarity and simplicity, not every element is labeled in every figure, nor is every element of each embodiment shown where illustration is not necessary to allow those of ordinary skill in the art to understand the disclosure. Moreover, reference characters may indicate elements in some figures which are illustrated in other figures, and which, for the sake of brevity, are described only with reference to the other figures.

The detailed description will be better understood in conjunction with the accompanying drawings, wherein like reference characters represent like elements, as follows:

FIG. 1 depicts an exploded perspective view of an illustrative mixing system for mixing an injectable material;

FIG. 2 depicts a cross-sectional view of the multi-reservoir system of the mixing system of FIG. 1, taken at line 2-2;

FIG. 3 depicts a perspective view of the illustrative multi-reservoir system with the cap removed;

FIG. 4 depicts a cross-sectional view of the multi-reservoir system coupled with the syringe;

FIG. 5 depicts a cross-sectional view of the multi-reservoir system coupled with the syringe in a dispensed configuration;

FIG. 6 depicts a side view of the illustrative multi-reservoir system being shaken;

FIG. 7 depicts a perspective view of the illustrative multi-reservoir system with the plug and syringe removed;

FIG. 8 depicts a side view of the illustrative multi-reservoir system as air is being purged;

FIG. 9A depicts an enlarge perspective view of the illustrative injection system;

FIG. 9B depicts a perspective view of an assembled illustrative injection system and saline syringe;

FIG. 9C depicts a perspective view of the unassembled illustrative injection system and saline syringe;

FIG. 10 depicts a side view of the unassembled injection system and multi-reservoir system;

FIG. 11 depicts a side view of the assembled injection system and multi-reservoir system;

FIG. 12 depicts a cross-sectional view of the assembled injection system and multi-reservoir system taken at line 12-12 of FIG. 11;

FIG. 13 depicts the cross-sectional view of FIG. 12 with the plunger assembly actuated to a partially dispensed configuration;

FIG. 14 depicts a cross-sectional view of the assembled injection system multi-reservoir system in a dispensed configuration; and

FIG. 15 depicts a cross-sectional view of the multi-reservoir system coupled with the syringe in an alternative configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location.

Various medical procedures involve delivery (e.g., injection) of injectable material(s) into the body before, during, or after the procedure. Preferably, the injectable material is biocompatible, and optionally biodegradable. The injectable material may serve a variety of purposes, including, without limitation, differentiating tissue (e.g., by creating a “bleb” or other raised or swelled region to distinguish an anatomical region), spacing anatomical structures from one another, otherwise affecting (e.g., shielding, coating, covering, modifying, etc.) an anatomical structure, etc. It will be appreciated that the term “tissue” is a broad term that encompasses a portion of or site within a body: for example, a group of cells, a group of cells and interstitial matter, an organ, a portion of an organ, an anatomical portion of a body, e.g., a rectum, ovary, prostate, nerve, cartilage, bone, brain, or portion thereof, etc. Moreover, reference may be made, herein, to a “target” in referring to an area of the patient's body at which a procedure is to be performed. However, it will be appreciated that such reference is to be broadly understood and is not intended to be limited to tissue or to particular procedures. Finally, reference may be made to target tissue, target location, target site, target tissue site, anatomical site, delivery site, deployment site, injection site, treatment site, etc., including combinations thereof and other grammatical forms thereof, interchangeably and without intent to limit.

Certain specific aspects of the present disclosure relate to placing an injectable material between target tissue to be treated and other tissues. For the sake of convenience, and without intent to limit, reference is made to an injectable material, such as a filler, including, without limitation, a gel composition. The injectable material may be delivered within the patient to displace the tissue relative to a tissue that is to be treated by a therapeutic procedure or otherwise (e.g., not necessarily therapeutic). Certain aspects of the present disclosure include displacing and/or shielding a tissue to protect the tissue against possible side effects of treatment of a target tissue, such as the effects of a treatment involving radiation or cryotherapy. In some aspects, the injectable material may displace anatomical tissue and/or may increase the distance between the target tissue and other tissues. For instance, if the target tissue is to be irradiated, the injectable material may space other tissues from the target tissue so that the other tissues are exposed to less radiation and/or are shielded from the radiation. In some aspects, the injectable material is injected as a filler in a space between tissues. A first tissue may then be treated by radiation, while the injectable material reduces passage of radiation therethrough into a second tissue. The first tissue may be irradiated while the second tissue, spaced by the injectable material, receives less radiation than it would have in the absence of the injectable material. An effective amount of an injectable material may be injected into a space between a first tissue to be treated and a second tissue which can be a critically sensitive organ. For instance, in the context of treatment of prostate cancer, an injectable material may be injected into the Denonvilliers' space (a region between the rectum and prostate) to create additional space between the rectum and prostate and/or to shield the rectum during treatment, thereby reducing rectal radiation dose and associated side effects.

In some aspects of the present disclosure, constituents of an injectable material are combined by a system formed in accordance with various principles of the present disclosure and injected into or near a target site. It will be appreciated that terms such as combine, mix, blend, etc., (including other grammatical forms thereof) may be used interchangeably herein without intent to limit unless otherwise indicated. Reference is accordingly made herein to a combining system generically, without intent to specifically require active combining/mixing.

In accordance with various principles of the present disclosure, the injectable material may be a filler such as a hydrophilic polymer, a gel, a hydrogel, etc. For instance, the injectable material can include polymeric materials which are capable of forming a hydrogel upon crosslinking. Optionally, the polymer forms a hydrogel within the body. A hydrogel is defined as a substance formed when a polymer (natural or synthetic) is crosslinked via covalent, ionic, or hydrogen bonds to create a three-dimensional structure which entraps water molecules to form a gel. Naturally occurring and synthetic hydrogel forming polymers, polymer mixtures and copolymers may be utilized as hydrogel precursors. In some aspects, the hydrogel can be formed by a composition formed by two or more constituents/components (e.g., mixing accelerant fluid, diluent, and polyethylene glycol (PEG) together), and may include one or more polysaccharide compounds or a salt thereof. For example, the composition may include a cellulose compound such as carboxymethyl cellulose (CMC) or salt thereof (e.g., CMC sodium), xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be crosslinked with a suitable crosslinking compound, such as butanediol diglycidyl ether (BDDE). In some aspects, the polysaccharide may be a homopolysaccharide or a heteropolysaccharide.

In some aspects of the present disclosure, two or more constituents of an injectable material are provided separately, and are combined by a device, system, and method in accordance with various principles of the present disclosure to form an injectable material to be injected into or near a target site by devices, systems, and methods in accordance with various principles of the present disclosure. The injectable material may be delivered within the patient to displace the tissue relative to a tissue that is to be treated by a therapeutic procedure or otherwise (e.g., not necessarily therapeutic). A composition to be injected into a patient may be a combination of two or more constituents combined by a device, system, or method formed in accordance with various principles of the present disclosure. In accordance with various principles of the present disclosure, a composition to be injected into a patient may be a combination of two or more constituents combined by a device, system, or method such as formed in accordance with various principles of the present disclosure. For instance, devices, systems, or methods of the present disclosure may be used to combine a first constituent and a second constituent for injection into a patient. The first constituent may be a precursor, e.g., a first constituent to be combined with an additional constituent to form the injectable compound. The second constituent may be an accelerator, an activating agent, a catalyst, an initiator, etc., which, upon combination with the precursor, produces the injectable compound, such as by altering the chemical composition or structure of the first constituent or precursor. The constituents may be combined prior (e.g., immediately prior) to delivery (e.g., injection) or as the constituents are being delivered to the patient so that the injectable material does not have time to form into a structure which may be difficult to inject or otherwise deliver to the patient. As such, the combination of the first constituent or precursor and the second constituent may be such that the injectable compound attains its desired properties and/or reaches its final form in situ.

In some embodiments, the injectable material is formed of a first constituent, a second constituent, and a third constituent. For instance, for various reasons it may be desirable to provide a first, precursor constituent in a solid form (e.g., to be more stable for storage and/or transport). The first constituent is combinable with the third constituent, and the thus-formed combined composition (which may be referenced as the precursor) is then combinable with the second constituent once the medical professional is ready to deliver (e.g., inject) the injectable material to the patient. The second constituent may facilitate a crosslinking interaction between the first and third constituents, for example, by initiating or accelerating the crosslinking interaction of the first and third constituents. Typically, one or more of the constituents of the injectable materials are biocompatible polymers. In some aspects, one of the first, second, or third constituents is a reactive polymer, such as a cross-linkable and/or hydrophilic polymer constituent (e.g., polyethylene glycol (PEG)), and one of the first, second, third constituent is a diluent (e.g., mostly water) in which a solid or semi-solid form of the reactive polymer is dissolved or dispersed, and/or with which the reactive polymer is cross-linked (or at least cross-linkable, such as upon further combination with the second constituent), to form a precursor. Another of the first, second, or third constituents may be an accelerator, an accelerant, an activating agent, a catalyst, an initiator, etc. (such terms being used interchangeably herein without intent to limit), combinable and reactive with the precursor to form the desired injectable material. In one example of an embodiment, a first constituent, in the form of a reactive polymer (specifically, PEG) that has been derivatized with reactive electrophilic groups (specifically, succinimide ester groups), is mixed with a third constituent, in the form of a cross-linking agent (specifically, trilysine, which contains multiple nucleophilic groups, specifically, amino groups), under acidic pH conditions where the succinimide ester groups and the amino groups do not react to any significant degree. When this mixture is combined with a second constituent, in the form of an accelerant (specifically, a basic buffer solution), the pH of the resulting mixture becomes basic, at which point the amino groups of the trilysine react with the succinimide ester groups of the PEG to form covalent bonds, thereby crosslinking the PEG and forming a hydrogel. It will be appreciated that reference to “first”, “second”, or “third” is not intended to connote a particular nature of the material or the order in which the material is combined. As such, “first”, “second”, and “third” may be used to reference any of three constituents forming an injectable material in accordance with various principles of the present disclosure. A non-limiting example of such constituents combinable by devices, systems, or methods in accordance with various principles of the present disclosure includes a reactive constituent, a diluent with which the reactive constituent is to be combined to form a precursor, and an accelerator combinable with the precursor to form an injectable material. The injectable material is a biocompatible material, such as a polymeric material, such as a filler, or such as a hydrogel.

In some examples, the composition may be or include a gel with a desired gel strength and/or viscosity, such as a biocompatible gel suitable for injection (e.g., through a needle), as discussed in further detail below. In one example of an embodiment, the first constituent is a biocompatible polymeric constituent. More particularly, in one example of an embodiment, the first constituent is a hydrophilic polymer, which may be natural or synthetic in origin, and may be anionic, cationic, zwitterionic, or neutrally charged. Non-limiting examples of hydrophilic polymers include natural hydrophilic polymers including proteins such as collagen and polysaccharides such as gellan gum, xanthan gum, gum arabic, guar gum, locust bean gum, alginate, and carrageenans, and synthetic hydrophilic polymers such as polyethylene glycols (PEG), PEG-methacrylates, PEG-methylmethacrylates, polyvinyl alcohols, polyacrylates and polymethacrylates, polyacrylic acids and their salts, polymethacrylic acids and their salts, polymethylmethacrylates, carboxymethylcelluloses, hydroxyethylcelluloses, polyvinylpyrrolidones, polyacrylamides such as N,N-methylene-bis-acrylamides or tris(hydroxymethyl)methacrylamides. The hydrophilic polymer may be modified to provide functional groups that are reactive with functional groups of a suitable cross-linking agent, which may be a covalent or ionic cross-linking agent.

The concentrations of gelling agent(s) in a composition formed in accordance with various principles of the present disclosure maybe at least about 0.01% by weight with respect to the total weight of the composition, and at most about 2.0% by weight with respect to the total weight of the composition, including increments of about 0.01% therebetween. For instance, the concentration of gelling agent(s) may range from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, a composition formed in accordance with various principles of the present disclosure may have a viscosity of at least about 0.001 pascal-second (Pa·s), and at most about 0.100 Pa·s at a shear rate of 130 s⁻¹. For instance, the composition may have a viscosity ranging from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, a composition formed in accordance with various principles of the present disclosure may have a viscosity of at least about 0.001 Pa·s, and at most about 0.050 Pa·s at a shear rate of 768 s⁻¹. For instance, the composition may have a viscosity ranging from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

In some embodiments, a multi-reservoir system includes separate reservoirs for constituents to be combined to form the injectable material to be delivered to the patient by an injection system. In some embodiments, a first constituent and a second constituent are separately contained within a first reservoir and a second reservoir, respectively, of a multi-reservoir device. A third constituent may be contained in a separate reservoir defining a third reservoir of the multi-reservoir system. To deliver the injectable material, the constituents of the first and third reservoirs are combined within the first reservoir (e.g., to form a precursor) and then the constituents of the first and second reservoirs are injected together into the patient. The multi-reservoir device may or may not mix the contents of the first reservoir with the contents of the second reservoir. For instance, the multi-reservoir device may deliver and inject the constituents of the injectable material to an injection system, with the injection system including a mixer component configured to mix the constituents from the first reservoir of the multi-reservoir device with the contents from the second reservoir of the multi-reservoir device as those contents are injected from the multi-reservoir device and the injection system into the patient. The already-combined first and third constituents are combined with the second constituent to form the desired form, structure, composition, properties, etc., of the injectable material to be delivered and deposited within the patient. The final form, structure, composition, properties, etc., of the injectable material may be attained once the combined constituents are within the patient.

The present disclosure provides devices, systems, and methods for combining constituents to form an injectable composition, and corresponding medical devices, systems, and methods for use thereof and/or delivery to a treatment site of a patient. According to some aspects of the present disclosure, such as described above, a multi-reservoir system may include a plurality of reservoirs for the one or more constituents of the injectable material and for combinations of such constituents. It will be appreciated that terms such as chamber, reservoir, container, vial, lumen, etc., may be used interchangeably herein without intent to limit, to refer to elements which contain, convey, hold, transport, collect, etc., a constituent (fluid, particulate, liquid, solid, gas, etc.) of an injectable material. Suitable chambers may include, for example, vials, syringes (e.g., a syringe barrel compatible with a manual or automatic injection system) and other fluid containers, such as configured for use with a suitable injection system. Examples of materials suitable for the reservoir of devices or systems of the present disclosure include, but are not limited to, cyclic olefin polymer, polypropylene, polycarbonate, polyvinyl chloride, and glass. In some aspects, one of these materials (e.g., cyclic olefin copolymer specifically) can have a coating applied to it (such as SiO₂ coating), which is advantageous so the coating can provide a primary oxygen barrier, behave as a glass-like layer, and/or can be applied using a vapor deposition process.

A combining device or system formed in accordance with various principles of the present disclosure to combine two or more constituents to form an injectable material may include and/or be removably connected to one or more injection systems which are configured to deliver the injectable materials to a patient. According to some aspects of the present disclosure, the filler compositions which may be used with various systems disclosed herein, e.g., the compositions prepared by the various devices, systems, methods disclosed herein, may have sufficient strength, e.g., gel strength, to withstand the forces on the continuity of the three-dimensional configuration (e.g., gel network) of the composition, and thereby minimize the effects of such forces. In the meantime, compositions with sufficient strength to withstand forces thereon may have a viscosity suitable for injection, e.g., a viscosity that does not cause the composition to become stuck in the reservoir(s), delivery lumen, needle, or other structure in which the composition is contained or through which it passes. According to some aspects of the present disclosure, the composition may maintain its three-dimensional structure until the composition is injected into a patient (e.g., through a needle), whereupon the structure may form fragments of the original continuous, three-dimensional network. Those fragments may have a diameter corresponding to the diameter of the lumen through which it passes into the patient (e.g., the lumen of an injection needle), such that the fragments are as large as possible in-vivo to retain as much of the three-dimensional structure of the composition as possible. Injection of these larger-sized particles or fragments is believed to increase the amount of time the gel remains within the tissue.

In some examples, the injection system includes a needle. In some embodiments, the needle may be a hypodermic needle, and may range from a size of 7-gauge (4.57 mm outer diameter (OD), 3.81 mm inner diameter (ID)) to 33-gauge (0.18 mm OD, 0.08 mm ID), e.g., a size of 16-gauge (1.65 mm OD, 1.19 mm ID), 18-gauge, 21-gauge (0.82 mm OD, 0.51 mm ID), 22-gauge (0.72 mm OD, 0.41 mm ID), 23-gauge (0.64 mm OD, 0.33 mm ID), or 24-gauge (0.57 mm OD, 0.31 mm ID). According to some aspects of the present disclosure, the size of the needle may be chosen based on the viscosity and/or constituents of the composition, or vice versa. According to some aspects of the present disclosure, the size of the needle may be 23-gauge or 25-gauge. In some cases, a larger size of 18-gauge, 20-gauge, 21-gauge, or 22-gauge may be used to inject the compositions disclosed herein. Examples of materials which may be used to form the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

As noted above, compositions used with systems disclosed herein may have large particulate matter (relative to the injection system lumen) and/or a high viscosity for passage through a lumen sized to inject the material into the patient. The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 pound-force (lb·f) to about 25 lb·f, such as from about 10 lb·f to about 20 lb·f, e.g., about 15 lb·f. The loads measured for a given gel concentration may vary for different needles and flow rates.

According to some aspects of the present disclosure, the size of the needle may be chosen based on the viscosity and/or constituents of the composition, or vice versa. According to some aspects of the present disclosure, the size of the needle may be 23-gauge or 25-gauge. In some cases, a larger size of 18-gauge, 20-gauge, 21-gauge, or 22-gauge may be used to inject the compositions disclosed herein.

According to some aspects of the present disclosure, a combining device or system can be included in a kit for introducing an injectable material into a patient, whereby the injectable material can include any of a variety of suitable compositions. Kits or systems may be configured to store one or more of the constituents of a composition until the medical professional is ready to mix the composition for delivery to a patient. For instance, compositions, such as hydrogels, may be prepared so that the precursor(s) and any related activating agent(s) are stored in the kit with diluents as may be needed. Applicators may be used in combination with the same. Kits formed in accordance with various principles of the present disclosure can be manufactured using medically acceptable conditions and contain constituents that have sterility, purity, and preparation that is pharmaceutically acceptable. Solvents/solutions may be provided in the kit or separately. The kit may include one or more syringes and/or needles for mixing and/or delivery of the injectable material, and/or for additional aspects of the procedure in which the injectable material is to be used. The kit or system may comprise various constituents as set forth herein. For instance, a target site into which an injectable material is to be delivered may be pre-treated using one or more constituents of the kit. One example of a pretreatment includes hydrodissection, such as with saline, to create space for injectable material to be injected at or in the vicinity of the target tissue site. Once saline has been injected to the treatment site, a combining device or system can be connected to a needle (e.g., an 18-gauge spinal needle) to then deliver the injectable material to the treatment site. For instance, in treating prostate cancer, a 5-10 mm layer of filler (e.g., gel composition) may be injected along the posterior wall of the prostate between the prostate and rectum. Once the filler has been injected into the space between the rectum and prostate, ultrasound images can be obtained.

In accordance with various principles of the present disclosure, a combining and/or delivery system is configured to facilitate combining of constituents of an injectable material. In some aspects, the injectable material is a combination of a first constituent, a second constituent, and a third constituent, such as described above. A combining and/or delivery system formed in accordance with various principles of the present disclosure facilitates combining/mixing of the various constituents of an injectable material. Additionally or alternatively, a combining and/or delivery system formed in accordance with various principles of the present disclosure facilitates delivery of the injectable material to an injection system configured to deliver (e.g., inject) and/or deposit the injectable material into a patient (e.g., to a target site within the patient). More particularly, various aspects of the present disclosure simplify assembly, alignment, mixing, dispensing, etc., of an injectable material which is combined or blended or mixed from separate constituents before delivery to a patient.

In some aspects, a combining and/or delivery system formed in accordance with various principles of the present disclosure includes two or more distinct lumens, chambers, or reservoirs for respective first and second constituents of an injectable material to be delivered and injected into a patient. A third constituent may be included in a separate device. The present disclosure facilitates combination of the separately-provided first and third constituents before a procedure, as well as combination of the recently-combined first and third constituents with the second constituent for injection into the patient. In particular, an example of an embodiment of a mixing and/or delivery system disclosed herein includes a combining system configured to facilitate the combining of the first, second, and third constituents of an injectable material before delivery to a patient. As described above, it may be desirable to provide the first, second, and third constituents separate from one another for combination only once a procedure is to be performed utilizing the injectable material formed by combining the first, second, and third constituents.

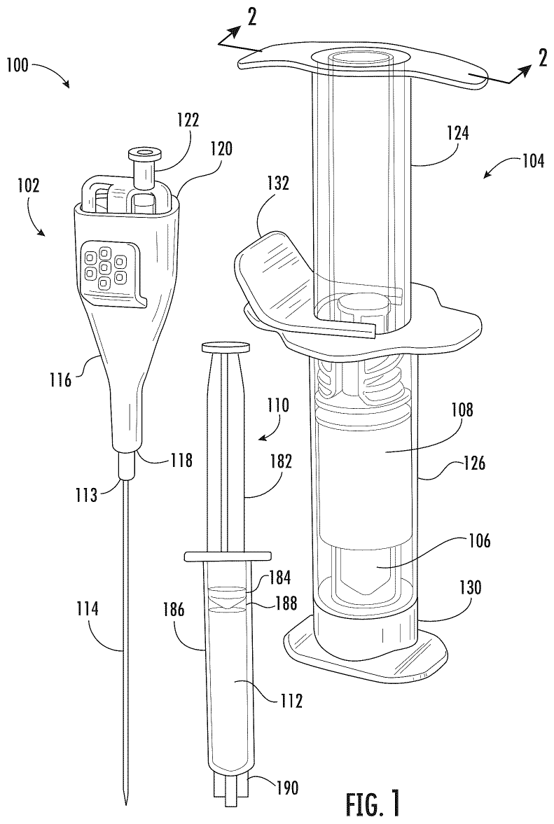

Turning to the drawings, FIG. 1 depicts an exploded perspective view of an illustrative combining and/or delivery system 100 in accordance with certain aspects of the present disclosure for mixing an injectable material. In some embodiments, the combining and/or delivery system 100 may be provided as a kit which may include, but is not limited to, a needle assembly or injection system 102 releasably attachable to a multi-reservoir system 104, a first constituent 106 disposed within a chamber or lumen of the multi-reservoir system 104, a second constituent 108 disposed within another chamber or lumen of the multi-reservoir system 104, a syringe 110, such as, but not limited to a diluent syringe, and a third constituent 112 disposed within a chamber or lumen of the syringe 110. The multi-reservoir system 104 may be used to transport the first and second constituents 106, 108 and the syringe 110 may be used to transport the third constituent 112 of an injectable material to a facility and/or location at which the injectable material is to be delivered into a patient. For the sake of convenience, and without intent to limit, reference may be made to delivery by injection into the patient (e.g., the injectable material is delivered to the patient by being injected into the patient), although the present disclosure need not be so limited. As described above, the first, second, and third constituents 106, 108, 112 may be mixed or combined at the time of treatment to form an injectable material.

The injection system 102 may include a needle 114 which can be any needle of this disclosure suitable for hydrodissection as well as delivering the injectable material (e.g., a gel composition) to the treatment site. A proximal end 113 of the needle 114 can be connected to a distal end 118 of a needle hub 116 (e.g., the needle 114 can be overmolded to connect to the needle hub 116). The proximal end 120 of the needle hub 116 may be attached to a distal end of a connector 122. In some embodiments, it is contemplated that the needle 114 may be replaced with a catheter tube or similar structure to reach a target location deeper in the body.

The syringe 110 may include a plunger 182, a plunger seal 184, a barrel 186 defining a lumen 188 extending therethrough, and a cap 190 disposed over a Luer lock (not explicitly shown in FIG. 1) adjacent to a distal end of the barrel 186. The plunger 182 and plunger seal 184 may be axially and/or longitudinally slidable with respect to the barrel 186 to push materials out of and/or to aspirate materials into the lumen 188 of the barrel 186. The cap 190 may provide a fluid-tight seal with a distal opening of the barrel 186 to allow fluid, such as the third constituent, or diluent 112 to be stored or pre-loaded within the lumen 188.

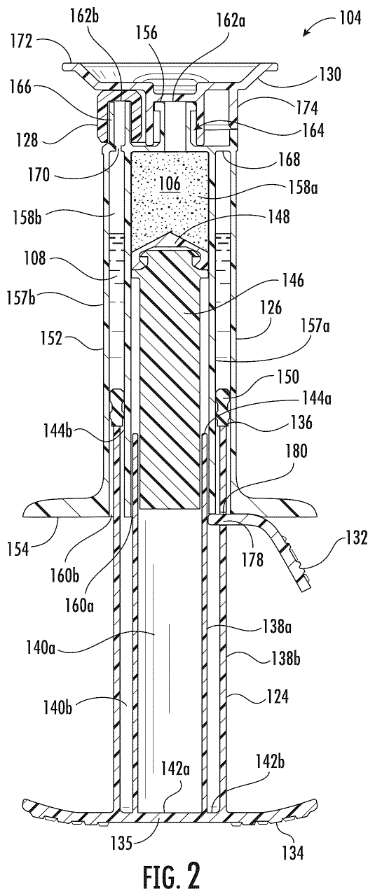

The multi-reservoir system 104 of the overall system 100 may generally include a plunger assembly 124, a barrel portion 126, a plug 128 (see, for example, FIG. 2), a cap 130, and a retainer 132. Briefly, a distal portion of the plunger assembly 124 may be slidably disposed within a proximal portion of the barrel portion 126. The barrel portion 126 may be configured to receive a third constituent 112 from the syringe 110 to first mix two constituents 106, 112 to form a precursor and then to mix the precursor with an accelerator 108 to form an injectable composition. In some cases, at least some of the mixing may occur within the multi-reservoir system 104 and some of the mixing may occur within the injection system 102, as will be described in more detail herein.

Referring additionally to FIG. 2, which depicts a cross-sectional view of the multi-reservoir system 104 taken at line 2-2 of FIG. 1, the plunger assembly 124 may extend from a generally planar proximal end 134 to a distal end 136. The proximal end 134 may define a flange or an actuation member 135 for depressing or actuating the plunger assembly 124. In some cases, a surface of the actuation member 135 may include texturing such as, but not limited to, raised bumps, ridges, etc. to improve the gripability of the proximal end 134. A first inner plunger 138a and a second outer plunger 138b may extend distally from actuation member 135 towards the distal end 136. The first plunger 138a may be generally tubular and may define a lumen or cavity 140a extending distally from a closed proximal end 142a to a distal opening 144a. Similarly, the second plunger 138b may be generally tubular and may define a lumen or cavity 140b extending distally from a closed proximal end 142b to a distal opening 144b. The first and second plungers 138a, 138b may be concentrically arranged to extend co-axially with the first plunger 138a disposed within a lumen or cavity 140b of the second plunger 138b. The first and second plungers 138a, 138b may have a generally circular outer shape. However, this is not required. Other shapes may be used as desired.

A third plunger 146 may be movably disposed relative to the plunger assembly 124. In some embodiments, the first plunger 138a may include a non-tubular structure configured to engage a lumen (not explicitly shown), if so provided, of the third plunger 146. For example, in some embodiments, the first plunger 138a may have a generally solid cross-section along at least a portion of the length thereof.

In some embodiments, the third plunger 146 may be movably disposed within at least a portion of the first lumen 140a. The third plunger 146 may float within the first lumen 140a. For example, the third plunger 146 may not be secured to either the plunger assembly 124 or the barrel portion 126. A distal end of the third plunger 146 may extend into a first reservoir 158a of the barrel portion 126. The third plunger 146 may include a stopper or seal 148. The seal 148 may be configured to provide a fluid tight seal between the third plunger 146 and a wall 157a of the first reservoir 158a of the barrel portion 126.

An annular seal 150 may be positioned adjacent to the distal end 136 of the second plunger 138b. The annular seal 150 may be coupled to the distal end 136 of the second plunger 138b or may be floating (e.g., uncoupled to the second plunger 138b) as desired. The annular seal 150 may be configured to provide a fluid tight seal between the second plunger 138b and a wall of a second reservoir 158b of the barrel portion 126.

In some embodiments, the plunger assembly 124 may be formed as a single monolithic structure. In other embodiments, the plunger assembly 124 may be formed as two or more distinct components that are subsequently coupled together. In some cases, the actuation member 135 and the first and second plungers 138a, 138b may be formed as a single monolithic structure and the third plunger 146, the seal 148, and the annular seal 150 may be provided as separate components. This is just one example. Other configurations may be used, as desired.

The barrel portion 126 may include a body portion 152 extending from a proximal end 154 to a distal end 156. In some examples, the proximal end 154 may include a flange configured to provide a gripping region to allow the user to actuate the plunger assembly 124 relative to the barrel portion 126. The body portion 152 may include an inner tubular wall 157a defining a first chamber, lumen, or reservoir 158a and an outer tubular wall 157b defining a second chamber, lumen, or reservoir 158b. The inner and outer tubular walls 157a, 157b may be concentrically arranged to extend co-axially with the inner tubular wall 157a disposed within the reservoir 158b of the outer tubular wall 157b. The inner and outer tubular walls 157a, 157b may have a generally circular outer shape. However, this is not required. Other shapes may be used as desired. In some embodiments, the barrel portion 126 may be formed as a single monolithic structure. In other embodiments, the barrel portion 126 may be formed as two or more distinct components that are subsequently coupled together.

In some examples, the inner tubular wall 157a may reduce in diameter towards the distal end 156 of the barrel portion 126 to form a first port 164. The first port 164 may be in fluid communication with the first reservoir 158a and a distal outlet 162a. The first port 164 may be configured to be releasably coupled to the syringe 110 as well as the injection system 102, as will be described in more detail here. The second reservoir 158b may include a distal wall 168 defining an aperture 170 extending therethrough. The aperture 170 may be in fluid communication with a second port 166 to fluidly couple the second reservoir 158b with a distal opening 162b. The second port 166 may be configured to be releasably coupled to the injection system 102, as will be described in more detail herein.

The first and second reservoirs 158a, 158b may be sized and shaped to receive at least a distal portion of the plunger assembly 124 which may be slidably disposed within the first and second reservoirs 158a, 158b of the barrel portion 126. For example, a distal end region of the first plunger 138a and a distal portion of the third plunger 146 may be disposed within the first reservoir 158a while a distal portion of the second plunger 138b and the seal 150 may be disposed within the second reservoir 158b.

In some embodiments, the cross-sectional dimension and/or shape of the first and second reservoirs 158a, 158b may decrease from the proximal end 154 to the distal end 156 of the barrel portion 126, although this is not required. The cross-sectional dimension may decrease in an abrupt step-wise manner to form discrete transitions in the cross-sectional dimension or the cross-sectional dimension may gradually taper.

The reservoirs 158a, 158b may be sized to hold a desired volume of respective constituents 106, 108. For example, the first constituent 106 may be held in the first, inner, reservoir 158a and the second constituent 108 may be held in the second, outer, reservoir 158b. The third plunger 146 and seal 148 may form a fluid tight seal at a proximal end region of the first reservoir 158a to allow a fluid to be stored in the first reservoir 158a while the cap 130 may selectively fluidly seal the distal opening 162a. In some examples, the cap 130 may be replaced with valve or other flow control mechanism configured to selectively fluidly seal the distal opening 162a. The annular seal 150 may form a fluid tight seal at a proximal end region of the second reservoir 158b to allow a fluid to be stored in the second reservoir 158b while the plug 128 may selectively fluidly seal the distal opening 162b. In some examples, the plug 128 may be replaced with valve or other flow control mechanism configured to selectively fluidly seal the distal opening 162b. In some examples, the volume of at least one of the reservoirs 158a, 158b may be increased or decreased as the constituents 106, 108 are moved within the multi-reservoir system 104.

A plug 128 may be releasably coupled to the second port 166 of the barrel portion 126. The plug 128 may be sized and shaped to be disposed over and fluidly seal the second distal opening 162b of the barrel portion 126. While not explicitly shown, the plug 128 may include an elastomeric or deformable sealing material disposed on an interior surface thereof and configured to contact the distal end of the second port 166 to form a fluid tight seal between the plug 128 and second port 166 of the barrel portion 126. The plug 128 may form a snap fit with the second port 166 of the barrel portion 126. However, other coupling mechanisms may be used, as desired, such as, but not limited to, friction fits, threaded engagements, rotational locks, etc.

A cap 130 may be releasably coupled to the distal end 156 of the barrel portion 126. The cap 130 may be sized and shaped to be disposed over and fluidly seal the first distal opening 162a of the barrel portion 126. The cap 130 may be eccentrically shaped to accommodate the plug 128. For example, a distal end 172 of the cap 130 may extend radially over a distal end of the plug 128 while a body portion 174 of the cap 130 is not disposed over the plug 128. Said differently, the plug 128 may be positioned exterior to a cavity defined by the body portion 174 of the cap 130. While not explicitly shown, the cap 130 may include an elastomeric or deformable sealing material disposed on an interior surface thereof and configured to contact the distal end 156 of the barrel portion 126 to form a fluid tight seal between the cap 130 and first port 164 of the barrel portion 126. The cap 130 may form a snap fit with the distal end 156 of the barrel portion 126. However, other coupling mechanisms may be used, as desired, such as, but not limited to, friction fits, threaded engagements, rotational locks, etc.

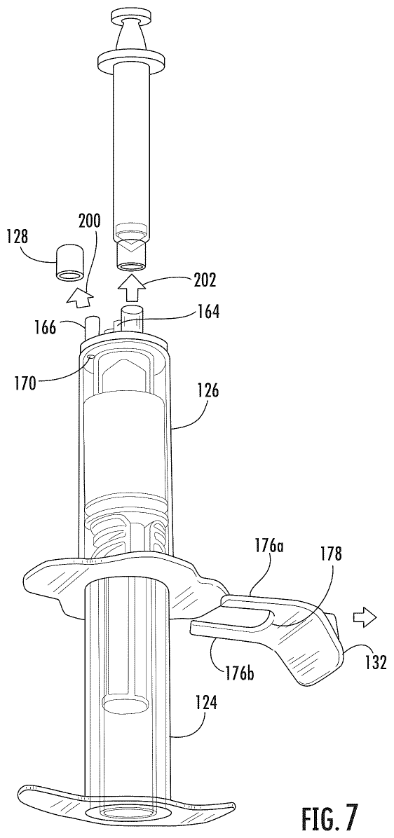

The retainer 132 may be removably positioned proximal to the proximal end 154 of the barrel portion 126. In some cases, the retainer 132 may form a snap fit, or other coupling mechanism with the plunger assembly 124. For example, the retainer 132 may include a pair of arms 176a, 176b (see, for example, FIG. 7) extending from a curved bridge region 178. The arms 176a, 176b may be configured to be positioned on opposing sides of the second plunger 138b while the curved bridge region 178 is received within a groove or recess 180 formed in the second plunger 138b. In other embodiments, the retainer 132 may form a friction fit with the plunger assembly 124. While the retainer 132 is positioned between the plunger assembly 124 and the barrel portion 126, distal actuation of the plunger assembly 124 and/or proximal actuation of the barrel portion 126 may be precluded.

Generally, the plunger assembly 124 and the barrel portion 126 may be assembled in a telescoping arrangement. For example, a portion of plunger assembly 124 may be disposed within a portion of the barrel portion 126. More particularly, the plunger assembly 124 may be assembled with the barrel portion 126 such that the first plunger 138a and the third plunger 146 of the plunger assembly 124 are slidably disposed within the first reservoir 158a of the barrel portion 126 and the second plunger 138b and annular seal 150 of the plunger assembly 124 is slidably disposed within the second reservoir 158b of the barrel portion 126. The plunger assembly 124 is movable, such as axially and/or longitudinally slidable, with respect to the barrel portion 126 to move (e.g., eject) materials out of and/or to move (e.g., aspirate) materials into first and/or second reservoirs 158a, 158b within the barrel portion 126. Similarly, the third plunger 146 may be axially and/or longitudinally slidable with respect to the first plunger 138a and the first reservoir 158a of the barrel portion 126 to allow materials out of and/or to move (e.g., aspirate) materials into first reservoir 158a within the barrel portion 126.

The barrel portion 126 may be pre-loaded with some of the constituents required to form the injectable material. For example, a first constituent, such as, but not limited to, a first cross-linkable constituent 106 may be disposed within the first reservoir 158a of the barrel portion 126. In some examples, the first cross-linkable constituent 106 may be provided as a powder. A second constituent 108, such as, but not limited to, an accelerator, may be disposed within the second reservoir 158b of the barrel portion 126. In some examples, the second constituent 108 may be provided as a liquid. A third constituent 112, such as, but not limited to, a second cross-linkable constituent, may be disposed within the lumen 188 of the syringe 110. In some examples, the third constituent 112 may be provided as a liquid. The third constituent 112 may be fluidly isolated from the first constituent 106 until mixing is desired. Further, the second constituent 108 may be fluidly isolated from each of the first and third constituents 106, 112 until mixing is desired.

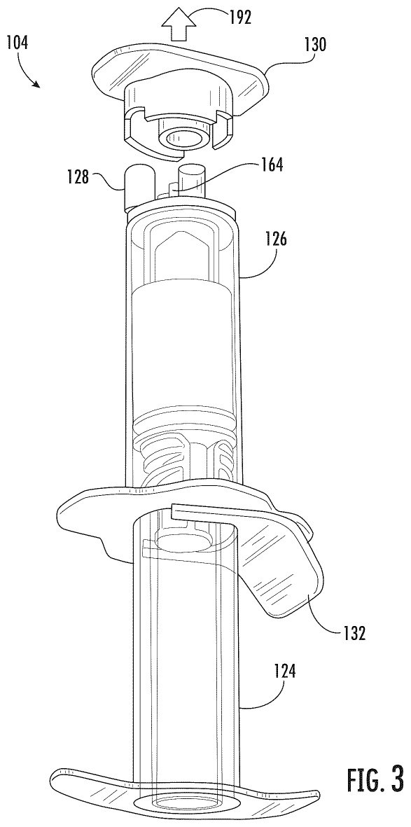

A method for dispensing or injecting the injectable material, along with additional features of the combining and/or delivery system 100 will now be described with respect to FIGS. 3-14. While certain steps are shown as a sequence between each figure, in other embodiments fewer steps are contemplated and the order by which steps are performed can be different than what is illustrated. To begin, the cap 130 is removed from the barrel portion 126, as shown in FIG. 3, which depicts a perspective view of the illustrative multi-reservoir system 104 with the cap 130 removed. The cap 130 may be axially displaced, as shown at arrow 192, to uncouple the cap 130 from the barrel portion 126. However, other movements or actions may be used to uncouple the cap 130 from the barrel portion 126.

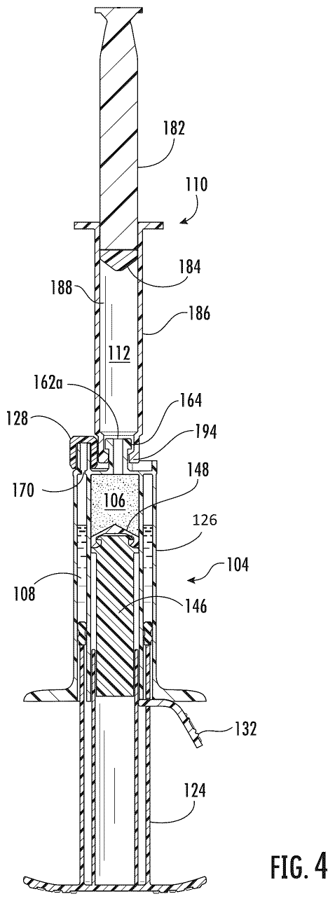

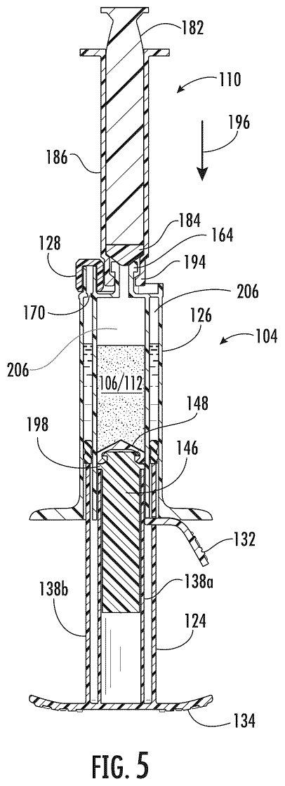

Once the cap 130 has been removed, the cap 190 of the syringe 110 may be also removed. In some cases, the cap 190 of the syringe 110 may be removed prior to removing the cap 130 of the multi-reservoir system 104. With both caps 130, 190 removed, the Luer lock 194 of the syringe 110 may be coupled with the first port 164 of the barrel portion 126, as shown in FIG. 4 which depicts a cross-sectional view of the multi-reservoir system 104 coupled with the syringe 110. Connecting the syringe 110 with the first port 164 may fluidly couple the lumen 188 of the syringe 110 with the first distal opening 162a and the first reservoir 158a. The plunger 182 of the syringe 110 may then be axially displaced, as shown in FIG. 5 which depicts a cross-sectional view of the multi-reservoir system 104 coupled with the syringe 110 in a dispensed configuration. As the plunger 182 of the syringe 110 is axially displaced towards the barrel portion 126, as shown at arrow 196, the third constituent 112 is dispensed from the lumen 188 of the syringe 110 into the first reservoir 158a of the barrel portion 126 via the first port 164. As the third plunger 146 floats within the first reservoir 158a of the barrel portion 126 and within the cavity 140a of the first plunger 138a, the third plunger 146 and the seal 148 may be axially displaced towards the proximal end 134 of the plunger assembly 124. Axial displacement of the third plunger 146 and the seal 148 may allow the volume of the first reservoir 158a to increase to accommodate the additional volume received from the syringe 110. The third plunger 146 may include a radially extending flange 198. The radially extending flange 198 may be configured to contact the distal end of the first plunger 138a to limit proximal axial movement of the third plunger 146 and seal 148. Some air 206 may remain in the first reservoir 158a of the barrel portion 126 and/or in the second reservoir 158b of the barrel portion 126 after injection of the third constituent 112 into the first reservoir 158a.

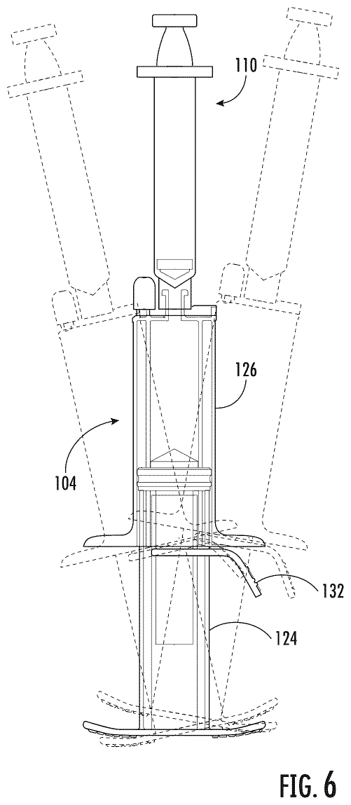

Once the third constituent 112 has been injected from the syringe 110 into the first reservoir 158a, the multi-reservoir system 104 may be shaken, as shown in FIG. 6, which illustrates a side view of the illustrative multi-reservoir system 104 and syringe 110 being shaken, to mix the first constituent 106 and the third constituent 112 to form the precursor 106/112. In some cases, the syringe 110 may remain coupled to the first port 164 as the multi-reservoir system 104 is shaken to prevent fluid from unintentionally spilling from the first distal opening 162a. In some cases, the syringe 110 may be removed and the cap 130 replaced to limit or prevent spilling. As used herein, the term “fluid” as it relates to constituents 106, 108, 112 of the system 100 is defined broadly and can include liquids, gels, oils and particulate matter such as granules, pellets, or powders, or any combination of liquids, gels, oils, and/or particulate matter (e.g., granules, pellets, or powders). As described above, in some examples, the third constituent 112 can be a diluent fluid solution and the first constituent 106 can include a cross-linkable polymer, such as PEG having a plurality of succinimidyl termini or any other agent mixable with diluent 112 to form precursor. The diluent can include a low molecular weight compound that contains multiple nucleophilic groups, such as trilysine which contains multiple amino groups, dissolved in a low pH (4.0) aqueous solution, though other diluent fluid solutions are contemplated within the scope of this disclosure. Once mixed together, the precursor solution 106/112 can be formed in the first reservoir 158a.

Next, the plug 128 and the syringe 110 may be removed as shown in FIG. 7 which depicts a perspective view of the illustrative multi-reservoir system 104 with the plug 128, syringe 110, and retainer 132 removed. In some examples, the plug 128 and/or syringe 110 may be axially displaced, as shown at arrows 200, 202. However, this is not required. In some examples, the plug 128 and/or syringe 110 may be removed with other movements and/or forces. Further, the retainer 132 may also be removed from the plunger assembly 124. The retainer 132 may be laterally displaced to uncouple the retainer 132 from the plunger assembly 124. However, other movements or actions may be used to uncouple the retainer 132 from the plunger assembly 124.

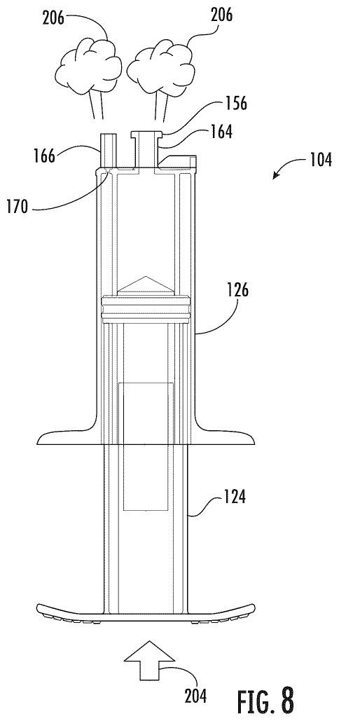

After the plug 128 and retainer 132 has been removed, the plunger assembly 124 may be actuated or distally advanced, as shown at arrow 204, to purge air 206 from the multi-reservoir system 104, as shown in FIG. 8, which depicts a side view of the illustrative multi-reservoir system 104, as air is being purged. The multi-reservoir system 104 may be held in an upright manner (e.g., with the distal end 156 of the barrel portion 126 pointed upwards (or away from the ground) to prevent an excess of fluid from dripping and/or leaking from the first and second reservoirs 158a, 158b of the barrel portion 126. In some cases, some fluid may be dispensed from the first and second reservoirs 158a, 158b during air purging to ensure no air remains in the first and/or second reservoirs 158a, 158b.

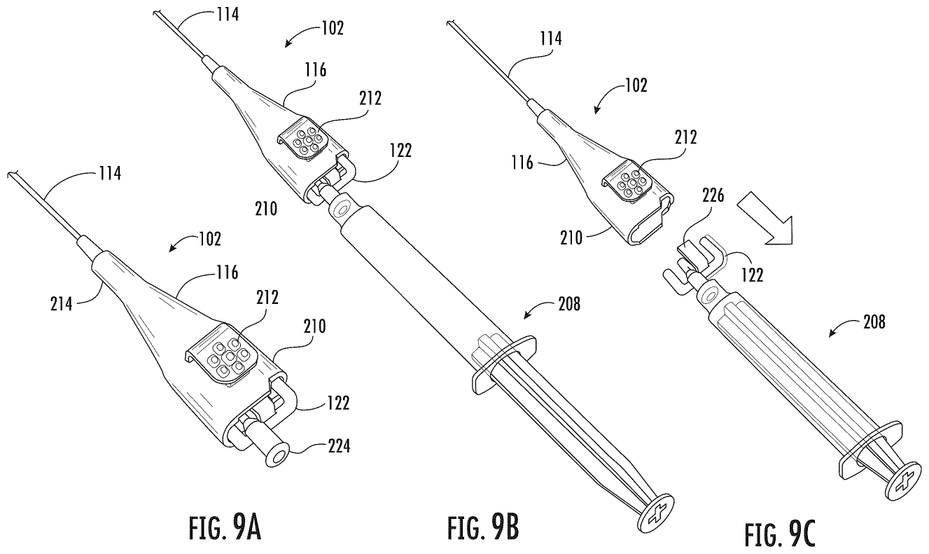

The multi-reservoir system 104 may be maintained in an upright orientation as hydrodissection is performed. Hydrodissection may be optionally performed before injecting the injectable material into the body. FIGS. 9A-9C depict an illustrative method for assembling and disassembling a syringe 208, such as, but not limited to, a saline syringe, for use in hydrodissection, with the injection system 102. Generally, the injection system 102 may be connected to the saline syringe 208. The needle 114 may then be positioned at the treatment site and saline injected to perform hydrodissection. In some examples, the saline syringe 208 may be provided separately from the combining and/or delivery system 100, although this is not required. Once the hydrodissection is complete, the saline syringe 208 may be uncoupled from the injection system 102 with the needle 114 remaining in position and primed at the treatment site (e.g., the distal end of the needle 114 remains in the body). However, this is not required. In some cases, the needle 114 may be removed from the body after hydrodissection.

Referring first to FIG. 9A, which depicts a perspective view of the illustrative injections system 102 and connector 122, the needle hub 116 may include a lower housing 210 configured to be gripped and squeezed by a user. One or more externally positioned buttons 212 can be positioned on an outer surface of lower housing 210. In some cases, two buttons 212 may be positioned on opposing sides of the lower housing 210. The button(s) 212 can be configured so that an actuating squeeze or other movement by a user causes latches of an adaptor or connector 122 and/or the barrel portion 126 of the multi-reservoir system 104 to release from mating apertures in the needle hub 116. However, other coupling mechanisms between the needle hub 116 and the connector 122 or barrel portion 126 are contemplated as needed or required. For example, and without limitation, snap fit connectors, magnetic connectors, female-male connectors, hook and loop fasteners, and the like are contemplated.

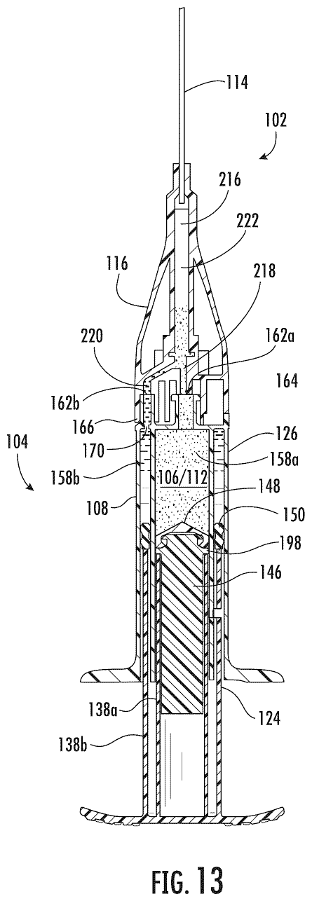

The needle hub 116 may also include a transitional portion 214 through which the needle 114 can be inserted. The transitional portion 214 can include a diameter (or cross-sectional dimension) smaller than the lower housing 210. In some examples, the transitional portion 214 can be tapered and/or include a textured outer surface. A central tubular lumen 216 connected to a first lumen 218 and a second lumen 220 (see, for example, FIG. 13) can pass through the needle hub 116 and be in fluid communication with the needle 114, when the needle 114 is connected to needle hub 116. The first and second lumens 218, 220 may be configured to be in fluid communication with one or more lumens of the connector 122 when connected thereto and to be in fluid communication with the distal openings 162a, 162b of the barrel portion 126 of the multi-reservoir system 104 when connected thereto. The connector 122 may be an adaptor, manifold, etc. configured to fluidly couple the single outlet of the saline syringe 208 to the first and second lumens 218, 220 of the needle hub 116. For example, the connector 122 may have a single fluid inlet for coupling to the outlet of the saline syringe 208 and two or more fluid outlets for coupling to first and second lumens 218, 220 of the needle hub 116. The connector 122 may include more than one fluid inlet or fewer than two or more than two fluid outlets as needed or desired. The first and second lumens 218, 220 of the needle hub 116 may intersect or merge at a mixing region 222 (see, for example, FIG. 13). The mixing region 222 may include a static mixer (not explicitly shown) configured to mix the fluids from the first and second lumens 218, 220.

To perform the hydrodissection, the injection system 102 and a saline syringe 208 may be required. While the syringe 208 is described as a saline syringe, the syringe 208 may include other fluids, as desired. To connect the injection system 102 with the saline syringe 208, a proximal end 224 of the connector 122, having a single fluid opening, may be aligned with the outlet of the saline syringe 208. The connector 122 may then be connected to the outlet of the saline syringe 208, as shown in FIG. 9B, which depicts a perspective view of the assembled illustrative injection system 102 and saline syringe 208. In some cases, the proximal end 224 of the connector 122 and the outlet of the saline syringe 208 may have mating Luer fittings. However, other connection mechanisms may be used as desired, such as, but not limited to, friction fits, snap fits, threaded engagements, etc. Hydrodissection may then be performed. Once the hydrodissection is complete, the saline syringe 208 may be uncoupled from the injection system 102 while leaving the needle 114 at the treatment site. It is further contemplated that the connector 122 may also be uncoupled from the injection system 102. In some cases, the connector 122 and the saline syringe 208 may be uncoupled from the injection system 102 substantially simultaneously. For example, actuation of the button 212 may cause one or more latches 226 of the connector 122 to release from a mating recess of the needle hub 116. In some cases, a latch 226 may be provided on each of the opposing sides of the connector 122, although this is not required. Proximal retraction of the saline syringe 208 while the button 212 of the needle hub 116 is actuated may release both the connector 122 and the saline syringe 208 from the needle hub 116, as shown in FIG. 9C, which depicts a perspective view of the unassembled illustrative injection system 102 and saline syringe 208. The connector 122 may remain coupled to the saline syringe 208 or may be subsequently uncoupled from the saline syringe 208, if so desired. While the connector 122 and the saline syringe 208 have been described as being uncoupled substantially simultaneously, in some cases, the saline syringe 208 may first be uncoupled from the connector 122 and then the connector 122 uncoupled from the needle hub 116.

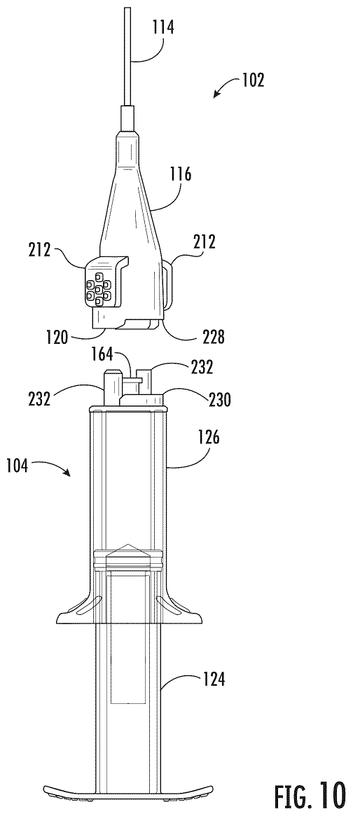

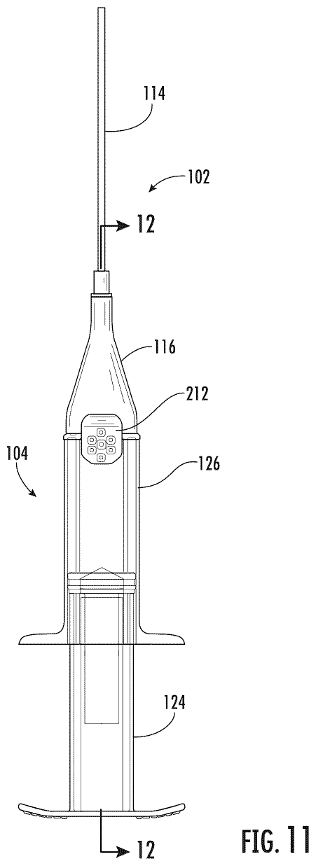

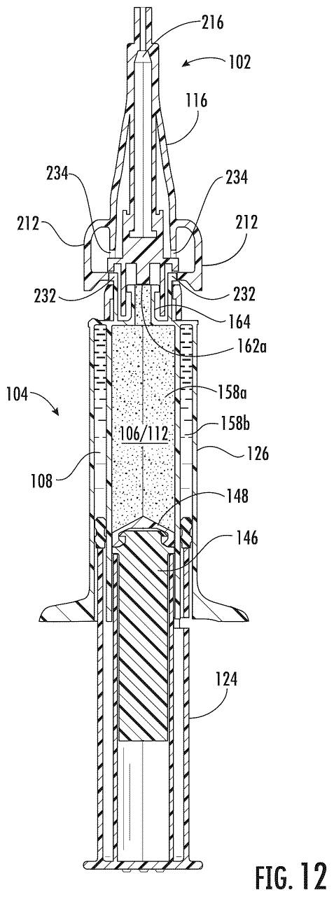

Next, the multi-reservoir system 104 may be connected to the needle hub 116. To do so, the first and second lumens 218, 220 of the needle hub 116 may be aligned with the distal openings 162a, 162b of the barrel portion 126, as shown in FIG. 10 which depicts a side view of the unassembled injection system 102 and multi-reservoir system 104. In FIG. 10, the second port 166 may be disposed behind the first port 164. In some cases, a cut-out 228 on a proximal end 120 of the needle hub 116 may be aligned with a lip or ridge 230 extending distally from a distal end region of the barrel portion 126, although this is not required. When assembled, the lower housing 210 of the needle hub 116 may be slid over the first and second ports 164, 166 of the barrel portion 126, as shown in FIG. 11 which depicts a side view of the assembled injection system 102 and multi-reservoir system 104. Referring additionally to FIG. 12 which depicts a cross-sectional view of the assembled injection system 102 (with the needle 114 removed for clarity) and multi-reservoir system 104 taken at line 12-12 of FIG. 11, when the needle hub 116 is assembled with the distal end region of the barrel portion 126, one or more tabs 232 of the barrel portion 126 may be received within one or more mating apertures 234 of the needle hub 116. The one or more apertures 234 may be disposed below the one or more buttons 212 such that actuating (e.g., depressing) the one or more buttons 212 is configured to disengage the one or more tabs 232 of the barrel portion 126 from the one or more mating apertures 234 to allow the injection system 102 to be uncoupled from the multi-reservoir system 104.

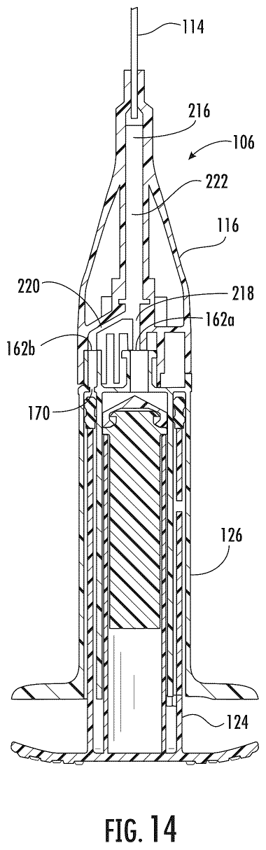

Once the multi-reservoir system 104 has been assembled with the injection system 102, the plunger assembly 124 can be actuated or distally advanced to cause the precursor fluid 106/112 (e.g., resulting from the mixture of the first constituent 106 and the third constituent 112) disposed in the first reservoir 158a of the barrel portion 126 and the second constituent 108 (e.g., the accelerator, such as, but not limited to a basic buffer solution) disposed in the second reservoir 158b of the barrel portion 126 to be dispensed from the distal openings 162a, 162b. Referring additionally to FIG. 13 which depicts a cross-sectional view of the assembled injection system 102 and multi-reservoir system 104 in a partially dispensed configuration, the first and second lumens 218, 220 of the needle hub 116 may be in fluid communication with the first and second reservoirs 158a, 158b and the distal openings 162a, 162b. As the plunger assembly 124 is actuated, distally advanced, or depressed, fluid may exit the first and second reservoirs 158a, 158b of the barrel portion 126 and enter the first and second lumens 218, 220 of the needle hub 116. For example, the distal end of the first plunger 138a may engage the radially extending flange 198 of the third plunger 146 to actuate or distally advance the third plunger 146 and seal 148 to expel fluid (precursor 106/112) from the first distal opening 162a. Similarly, the second plunger 138b and annular seal 150 may actuate or distally advance to expel fluid (second constituent 108) from the second distal opening 162b. The first and second lumens 218, 220 of the needle hub 116 may be connected with a mixing region 222. The mixing region 222 may be configured to mix or combine the precursor 106/112 and the second constituent 108 prior to the constituents exiting the needle 114. For example, the mixing region 222 may mix or combine the precursor and the third constituent 112 to form the injectable composition before the resulting mixture enters the central lumen 216 of the needle hub 116 which in turn is in fluid communication with a lumen of the needle 114. The injectable composition may continue egressing through the needle 114 and ultimately to the treatment site. The injectable composition formed from the combination of the precursor 106/112 and the second constituent 108 may attain its final desired properties and/or reach its final form in situ or at the target site. In the fully dispensed configuration, as shown in FIG. 14, the annular seal 150 of the second plunger 138b may contact the distal wall 168 of the barrel portion 126, although this is not required.

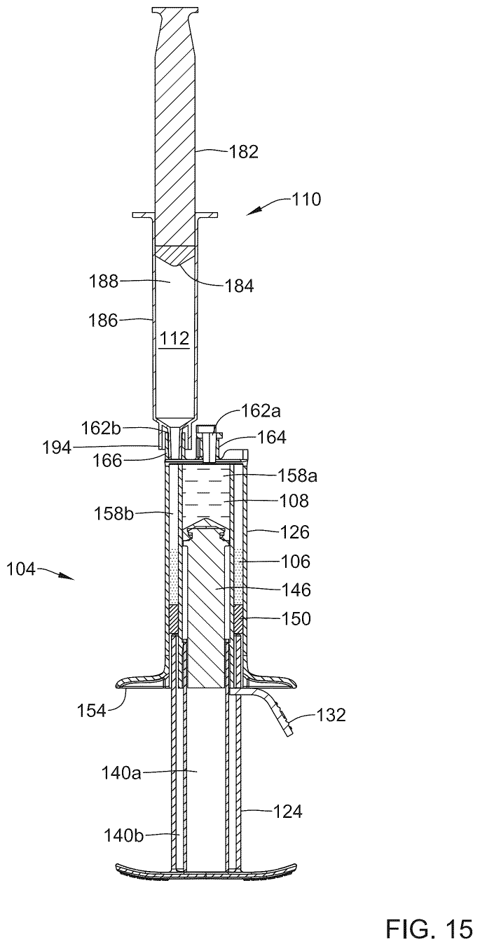

It is contemplated that while the first constituent 106 is shown and described as being disposed within the central first reservoir 158a and the second constituent 108 is shown and described as being disposed within the annular second reservoir 158b, the configuration may be reversed. FIG. 15 depicts a cross-sectional view of the multi-reservoir system 100 coupled with the syringe 110 and having the first and second constituents 106, 108 in an alternative configuration. The first constituent 106 is disposed within the second reservoir 158b and the second constituent 108 is disposed within the first reservoir 158a. Structurally, the multi-reservoir system 104 may remain the same with only the placement of the first and second constituents 106, 108 changed.

The third plunger 146 and seal 148 may form a fluid tight seal at a proximal end region of the first reservoir 158a to allow a fluid to be stored in the first reservoir 158a while the cap 130 may selectively fluidly seal the distal opening 162a. In some examples, the cap 130 may be replaced with valve or other flow control mechanism configured to selectively fluidly seal the distal opening 162a. For example, the cap 130 may be replaced with a plug (similar in form and function to plug 128) configured to be releasably coupled the first port 164. While not explicitly shown, such a plug may be sized and shaped to be disposed over and fluidly seal the first distal opening 126a of the barrel portion 126. The annular seal 150 may form a fluid tight seal at a proximal end region of the second reservoir 158b to allow a fluid to be stored in the second reservoir 158b while the plug 128 may selectively fluidly seal the distal opening 162b. In some examples, the plug 128 may be replaced with valve or other flow control mechanism configured to selectively fluidly seal the distal opening 162b. In some examples, the volume of at least one of the reservoirs 158a, 158b may be increased or decreased as the constituents 106, 108 are moved within the multi-reservoir system 104.

The method for dispensing or injecting the injectable material may be similar to that described with respect to FIGS. 3-14. When the first cross-linkable constituent 106 is disposed within the second reservoir 158b, the cap 130 and the plug 128 may be removed from the barrel portion 126. In cases, where the cap 130 is replaced with a plug, the plug covering the first port 164 may remain in place while the plug 128 covering the second port 166 is removed.

Once the plug 128 and/or the cap 130 have been removed, the cap 190 of the syringe 110 may be also removed. In some cases, the cap 190 of the syringe 110 may be removed prior to removing the plug 128 and/or the cap 130 of the multi-reservoir system 104. With both the plug 128 and the cap(s) 130, 190 removed, the Luer lock 194 of the syringe 110 may be coupled with the second port 166 of the barrel portion 126, as shown in FIG. 15. Connecting the syringe 110 with the second port 166 may fluidly couple the lumen 188 of the syringe 110 with the second distal opening 162b and the second reservoir 158b. The plunger 182 of the syringe 110 may then be axially displaced towards the barrel portion 126. The third constituent 112 is dispensed from the lumen 188 of the syringe 110 into the second reservoir 158b of the barrel portion 126 via the second port 166. In some cases, the plunger assembly 124 and the annular seal 150 may be axially displaced in a direction away from the second port 166 as the third constituent 112 is dispensed into the second reservoir 158b. Axial displacement of the plunger assembly 124 and the annular seal 150 may allow the volume of the second reservoir 158b to increase to accommodate the additional volume received from the syringe 110. While not explicitly shown, the plunger assembly 124 may include a mechanical interlock configured to engage a mating mechanical interlock on the barrel portion 126 to limit axial movement of the plunger assembly 124. Some air may remain in the first reservoir 158a of the barrel portion 126 and/or in the second reservoir 158b of the barrel portion 126 after injection of the third constituent 112 into the second reservoir 158b.

Once the third constituent 112 has been injected from the syringe 110 into the second reservoir 158b, the multi-reservoir system 104 may be shaken, to mix the first constituent 106 and the third constituent 112 to form the precursor 106/112. In some cases, the syringe 110 may remain coupled to the second port 166 as the multi-reservoir system 104 is shaken to prevent fluid from unintentionally spilling from the first distal opening 162a. In some cases, the syringe 110 may be removed and the plug(s) 128 and/or cap 130 replaced to limit or prevent spilling. After mixing, any of the syringe 110, plug(s) 128, and/or cap 130 that are secured to the barrel portion 126 may be removed. The method for mixing and injecting the precursor 106/112 and the second constituent 108 may continue as described with respect to FIGS. 8-14 with the only difference being the location of the precursor 106/112 (e.g., in the second reservoir 158b) and the second constituent 108 (e.g., in the first reservoir 158a).

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A system for producing a mixture to deliver to a treatment site, comprising:
a needle hub;
a multi-reservoir system, the multi-reservoir system comprising:
a plunger assembly including a first tubular plunger and a second tubular plunger;
a barrel portion including a housing including an inner tubular wall defining a first reservoir in fluid communication with a first port and configured to contain a first constituent and an outer tubular wall together with the first wall defining a second annular reservoir in fluid communication with a second port and disposed about the first reservoir, the second annular reservoir configured to contain a second constituent; and
a third plunger movably disposed relative to the plunger assembly and at least partially within the first reservoir; and
a syringe defining a lumen configured to contain a third constituent;
wherein a distal end of the syringe is configured to be releasably coupled to the first port or the second port such that actuation of the syringe causes the third constituent to be injected into the first reservoir or the second reservoir to mix with the first or second constituent and form a precursor; and
wherein actuating the plunger assembly relative to the barrel portion causes the precursor containing the first and third constituents and the second constituent to be delivered into the needle hub or causes the precursor containing the second and third constituents and the first constituent to be delivered into the needle hub.

2. The system of claim 1, further comprising a removable retainer positioned between a proximal end of the barrel portion and a proximal end of the plunger assembly, the removable retainer preventing actuation of the plunger assembly relative to the barrel portion.

3. The system of claim 1, further comprising a cap removably coupled with the first port of the barrel portion.

4. The system of claim 1, further comprising a plug removably coupled with the second port of the barrel portion.

5. The system of claim 1, wherein the first plunger and the second plunger are concentrically arranged.

6. The system of claim 1, wherein the first reservoir and the second annular reservoir are concentrically arranged.

7. The system of claim 4, wherein the plunger assembly is configured to be actuated with the first port of the barrel portion free from the cap and the second port of the barrel portion free from the plug.

8. The system of claim 1, wherein the third plunger is unsecured to both the plunger assembly and the barrel portion.

9. The system of claim 1, further comprising a needle that is configured to be coupled to the needle hub.

10. The system of claim 1, wherein the needle hub comprises a first lumen in fluid communication with the first reservoir of the barrel portion via the first port, a second lumen in fluid communication with the second reservoir of the barrel portion via the second port, a central lumen configured to be in fluid communication with a needle, and a mixing region connecting the first and second lumens with the central lumen.

11. The system of claim 1, wherein the needle hub is removably coupled to a distal end region of the barrel portion of the multi-reservoir system.

12. The system of claim 1, wherein the plunger assembly and the barrel portion are assembled in a telescoping arrangement.

13. A kit for producing a mixture for delivery to a treatment site, the kit comprising:
a multi-reservoir system, the multi-reservoir system comprising:
a plunger assembly including a first tubular plunger and a second tubular plunger;
a barrel portion including a housing including an inner tubular wall defining a first reservoir in fluid communication with a first port and configured to contain a first constituent and an outer tubular wall together with the first wall defining a second annular reservoir in fluid communication with a second port and disposed about the first reservoir, the second annular reservoir configured to contain a second constituent; and
a third plunger movably disposed relative to the plunger assembly and at least partially within the first reservoir;
a syringe defining a lumen configured to contain a third constituent; and
an injection system, the injection system comprising:
a needle hub; and
a needle coupled to the needle hub.

14. The kit of claim 13, further comprising a removable retainer positioned between a proximal end of the barrel portion and a proximal end of the plunger assembly, the removable retainer configured to prevent actuation of the plunger assembly relative to the barrel portion.

15. The kit of claim 13, further comprising a cap removably coupled with the first port of the barrel portion.

16. The kit of claim 13, further comprising a plug removably coupled with the second port of the barrel portion.

17. The kit of claim 13, further comprising a connector, the connector configured to couple the needle hub to a saline syringe.

18. A method for producing a mixture with a mixing system to deliver to a treatment site, the method comprising:
coupling a distal end of a syringe with a first port of the multi-reservoir system;
injecting a first constituent into a first reservoir of the multi-reservoir system to mix the first constituent with a second constituent contained in the first reservoir of the multi-reservoir system to form a precursor;
coupling a needle hub having a mixing region to a distal end of the multi-reservoir system; and
actuating a plunger assembly to move the precursor and a third constituent disposed within a second reservoir of the multi-reservoir system into the mixing region of the needle hub to form an injectable mixture.

19. The method of claim 18, further comprising removing a retainer from the plunger assembly prior to actuating the plunger assembly.

20. The method of claim 18, further comprising actuating the plunger assembly prior to coupling the needle hub to the distal end of the multi-reservoir system to purge air from the multi-reservoir system.
